(19) United States
(12) Patent Application Publication
(10) Pub. No.: US 2024/0370947 A1
(43) Pub. Date: Nov. 7, 2024

(54) METHOD OF EVALUATING EFFECTIVE GREEN WATER RESOURCES BASED ON DYNAMIC CROP COEFFICIENTS AND DEVICE THEREOF

(71) Applicant: CHANGJIANG RIVER SCIENTIFIC RESEARCH INSTITUTE OF CHANGJIANG WATER RESOURCES COMMISSION, Wuhan City (CN)

(72) Inventors: Jijun Xu, Wuhan City (CN); Mingzhi Yang, Wuhan City (CN); Yongqiang Wang, Wuhan City (CN); Zhe Yuan, Wuhan City (CN); Sinuo Li, Wuhan City (CN); Jinhui Dai, Wuhan City (CN)

(21) Appl. No.: 18/382,224

(22) Filed: Oct. 20, 2023

(30) Foreign Application Priority Data
May 6, 2023 (CN) 202310517442.2

(51) Int. Cl. G06Q 50/02; G01N 33/24

(52) U.S. Cl. CPC G06Q 50/02; G01N 33/246; G01N 33/245

(57) ABSTRACT

The present disclosure relates to a method of evaluating effective green water resources based on dynamic crop coefficients and a device thereof. The method includes constructing a SWAT model of an area to be measured; constructing a mathematical relation expression of vegetation potential transpiration, and introducing dynamic crop coefficients into the SWAT model; evaluating results obtained by operating the SWAT model according to the measured evaporation data and leaf area index monitoring data obtained in advance, and calibrating crop growth parameters and evapotranspiration parameters; judging evaporation effectiveness according to a land cover type and a dynamic coverage calculation method, and carrying out daily simulation to obtain a total amount of effective green water resources in the area to be measured.

### CROSS-REFERENCE TO RELATED APPLICATION

This application claims the benefit and priority from Chinese Patent Application No. 202310517442.2 filed on May 6, 2023, the contents of which are herein incorporated by reference.

### TECHNICAL FIELD

The present disclosure relates to the technical field of water resources evaluation, in particular to a method of evaluating effective green water resources based on dynamic crop coefficients and a device thereof.

### BACKGROUND

Green water is the water consumed by forests, grasslands, wetlands and farmland through evapotranspiration, which is the main water source to support terrestrial ecosystems and rain-fed agricultural production. On a global scale, green water accounts for 65% of the total precipitation, while the traditional water resource (blue water) only accounts for 35%. Broadening the scope of water resources evaluation and including green water, which accounts for 65% of the total precipitation, into the water resource evaluation system will make the water resource evaluation system more reasonable and comprehensive, help to maintain an appropriate ratio of blue water to green water, and provide more effective guidance for more effective utilization and scientific management of water resources.

Solar radiation, air humidity, temperature and wind speed are important factors affecting the evaporation process. Therefore, Penman-Monteith formula with strong applicability is generally used to calculate evapotranspiration in the evaluation of green water resources. This is the reason why most distributed hydrological models use the Penman-Monteith formula to simulate evapotranspiration. Moreover, the distributed hydrological models not only can take the spatial variability of various influencing factors into full account, but also can simulate the vegetation growth process, reveal a green water transformation mechanism, and make it possible to simulate and quantitatively divide green water at different temporal and spatial scales. However, in reality, vegetation evapotranspiration will be different from reference evapotranspiration because of factors such as a leaf structure, stomatal characteristics, aerodynamic properties and root growth of different vegetation types. The Penman-Monteith formula only takes the influence of meteorological conditions on crop evapotranspiration into account, that is, reference evapotranspiration, but does not takes the characteristics of specific crops into account. The actual evapotranspiration of different vegetation types in their respective growth periods cannot be accurately reflected, so that hydrological models have shortcomings in green water simulation.

In addition, the previous water resources evaluation mostly focused on the blue water resources that can meet the consumption of social and economic systems, ignoring the green water resources (evapotranspiration) that can be used by crops. The traditional water resources evaluation cannot fully reflect the full connotation of water resources, resulting in the lack of hierarchy in the caliber of the water resources evaluation and directly leading to the huge difference in the water resources evaluation amount. Therefore, green water should be incorporated in the scope of water resources evaluation. The circular supply of green water to the terrestrial ecosystems reflects the water consumption of the soil-vegetation ecosystem in nature, including effective green water and ineffective green water. Only the green water resources that are directly used by human production activities in the circulation process, directly participate in the production process of economic quantity and ecological quantity or provide effective environmental services for related ecological subjects are effective. Other green water has no economic and social value for human beings, and such green water is ineffective. Identifying the effective green water resources that can produce a value to the area truly meets the purpose of water resources evaluation.

Generally speaking, canopy interception evaporation, water surface evaporation and vegetation transpiration are all regarded as effective green water because they participate in the physiological process of plants. Soil evaporation of the land use type such as sandy land, Gobi, saline-alkali land, swamp, bare land, bare rock gravel land and sparse grassland belongs to ineffective green water. The soil evaporation between plants is the most important, which is divided according to the vegetation coverage. In the previous method, according to the different growth stages of vegetation, the effective green water was calculated by multiplying the amount of green water by a fixed coverage coefficient. However, the coverage is constantly changing with the growth of vegetation, and the division method based on static coverage is likely to lead to a large error in the division of effective green water.

Therefore, the current water resources evaluation methods still have shortcomings in green water simulation and easily lead to a large error in the division of effective green water, which is not conducive to the effective utilization and scientific management of green water resources.

### SUMMARY

In view of this, the purpose of the present disclosure is to provide a method of evaluating effective green water resources based on dynamic crop coefficients and a device thereof, so as to solve the problems in the prior art that there are still shortcomings in green water simulation and it is easy to lead to a large error in the division of effective green water.

According to the first aspect of the embodiment of the present invention, a method of evaluating effective green water resources based on dynamic crop coefficients, comprising:
Dividing and extracting an area to be measured to obtain watershed information, and dividing hydrological response units according to the watershed information;
Constructing a SWAT model according to the hydrological response units and meteorological data, reservoir data and farmland management data obtained in advance;
Constructing a mathematical relation expression of vegetation potential transpiration according to crop information, soil information and meteorological information obtained in advance, and optimizing a calculation formula of vegetation transpiration in the SWAT model according to the expression to simulate dynamic crop coefficients;
Evaluating evapotranspiration results and crop leaf area index simulation results obtained by operating the SWAT model according to the measured evaporation data and leaf area index monitoring data obtained in advance, and calibrating crop growth parameters and evapotranspiration parameters in the SWAT model according to the evaluation result;
Judging evaporation effectiveness according to a land cover type and a dynamic coverage calculation method;
Using the SWAT model to simulate vegetation coverage every day according to the judgment result, and obtaining a total amount of effective green water resources in the area to be measured according to the simulation result.

Preferably, dividing and extracting an area to be measured to obtain watershed information and dividing hydrological response units according to the watershed information comprises:
Importing raster DEM data of the area to be measured into a geographic information system platform to obtain natural sub-watershed division data and river network water system data as watershed information;
Importing the watershed information into an initial SWAT model, and dividing hydrological response units according to a land use type, a soil type and a slope type.

Preferably, constructing a mathematical relation expression of vegetation potential transpiration according to crop information, soil information and meteorological information obtained in advance comprises:

The mathematical relation expression of the vegetation potential transpiration is as follows:

$$EP = \{ \begin{matrix}{ET}_{0} \cdot {K}_{cini} & phuc \leq {fr}_{phu 1} \\ {ET}_{0} \cdot {K}_{cmid} & {fr}_{phu 1} < phuc \leq {fr}_{phu 2} \\ {ET}_{0} \cdot {K}_{cend} & phuc > {fr}_{phu 2}\end{matrix}$$

Where EP represents the vegetation potential transpiration; ET₀ represents reference evapotranspiration; K_{c ini}, K_{c mid} and K_{c end} represent crop coefficients of vegetation in an initial growth period, a rapid growth period and a late growth period, respectively; phuc represents a unit percentage of plant heat accumulation; fr_{gw1} and fr_{gw2} represent an accumulated temperature ratio corresponding to a first point and a second point on an optimal leaf area index curve, respectively.

Preferably, evaluating evapotranspiration results and crop leaf area index simulation results obtained by operating the SWAT model according to the measured evaporation data and leaf area index monitoring data obtained in advance comprises:

Based on the measured evaporation data and the leaf area index monitoring data obtained in advance, selecting a correlation coefficient and a Nash-Sutcliffe efficiency coefficient to evaluate the evapotranspiration results and the crop leaf area index simulation results obtained by operating the SWAT model.
Preferably, the calibrated crop growth parameters comprise: the accumulated temperature ratio corresponding to the first point on the optimal leaf area index curve, the accumulated temperature ratio corresponding to the second point on the optimal leaf area index curve, a leaf area ratio corresponding to the first point on the optimal leaf area index curve, a leaf area ratio corresponding to the second point on the optimal leaf area index curve, and an accumulated temperature ratio corresponding to a potential maximum leaf area index and the time when an leaf area index begins to decline;
The calibrated evapotranspiration parameters comprise: a soil evaporation compensation factor, a plant absorption compensation factor, a shallow groundwater re-evaporation coefficient, a threshold depth of shallow aquifer “re-evaporation” or infiltration into a deep aquifer, an effective water capacity of a soil layer and a vegetation interception capacity.

Preferably, judging evaporation effectiveness according to a land cover type and a dynamic coverage calculation method comprises:
Judging canopy interception evaporation, residential building interception evaporation, swamp or water area evaporation, phreatic water evapotranspiration and water participating in vegetation transpiration as effective green water resources;
Using a dynamic coverage calculation method to calculate vegetation coverage of sparse forest land and non-high density grassland;
Calculating soil evaporation between vegetation according to the vegetation coverage of sparse woodland and non-high density grassland.

Preferably, the formula of the dynamic coverage calculation method is as follows:

$${a}_{i} = {LAI}_{i} / {LAI}_{mx}$$

Where aᵢ represents vegetation coverage on an i-th day of a vegetation growing season; LAIᵢ represents a leaf area index of the i-th day of a vegetation growing season; LAIₘₓ indicates a maximum leaf area index during a vegetation growth period.

Preferably, using the SWAT model to simulate vegetation coverage every day and obtaining a total amount of effective green water resources in the area to be measured according to the simulation result comprises:

Obtaining a total amount of effective green water resources in the area to be measured by the following formula:

$${Wgreen}_{a} = {\sum }_{i = 1}^{N} [ {W}_{can , i} + {Wwet}_{i} + {Wwt}_{i} + {Wgw}_{i} + {\sum }_{j = 1}^{M} ( {Wep}_{ij} + {a}_{ij} \cdot {Wes}_{ij} ) ]$$

Where Wgreenₐ represents a total amount of effective green water resources in the area; W_{can,i} represents an intercepted evaporation capacity on the i-th day; Wwetᵢ indicates a wetland evaporation capacity on the i-th day; Wwti indicates a water area evaporation capacity on the i-th day; Wgwᵢ represents a phreatic water evaporation capacity on the i-th day; Wepᵢⱼ represents a vegetation transpiration capacity of a j-th vegetation on the i-th day; Wesᵢⱼ represents a soil evaporation capacity of the j-th vegetation on the i-th day; W_{un,i} represents a soil evaporation capacity of unused land on the i-th day; aᵢⱼ indicates vegetation coverage of the j-th vegetation on the i-th day; M represents a vegetation type in the watershed; and N represents a total number of days in a year.

Preferably, further comprising: obtaining a total amount of ineffective green water resources in the area to be measured by the following formula:

$${Wgreen}_{u} = {\sum }_{i = 1}^{N} [ {W}_{un , i} + {\sum }_{j = 1}^{M} ( 1 - {a}_{ij} ) \cdot {Wes}_{ij} ]$$

Where Wgreenᵤ represents a total amount of ineffective green water resources in the area; W_{un,i} represent a soil evaporation capacity of the unused land on the i-th day.

According to the second aspect of the embodiment of the present invention, a device of evaluating effective green water resources based on dynamic crop coefficients, comprising:
A model constructing module, which is configured to divide and extract an area to be measured to obtain watershed information, divide hydrological response units according to the watershed information, and construct a SWAT model according to the hydrological response units and meteorological data, reservoir data and farmland management data obtained in advance;
An optimizing module, which is configured to construct a mathematical relation expression of vegetation potential transpiration according to crop information, soil information and meteorological information obtained in advance, and optimize a calculation formula of vegetation transpiration in the SWAT model according to the expression to simulate dynamic crop coefficients;
A parameter calibrating module, which is configured to evaluate evapotranspiration results and crop leaf area index simulation results obtained by operating the SWAT model according to the measured evaporation data and leaf area index monitoring data obtained in advance, and calibrate crop growth parameters and evapotranspiration parameters in the SWAT model according to the evaluation result;
A judging module, which is configured to judge evaporation effectiveness according to a land cover type and a dynamic coverage calculation method;
A result generating module, which is configured to use the SWAT model to simulate vegetation coverage every day according to the judgment result, and obtain a total amount of effective green water resources in the area to be measured according to the simulation result.

The technical scheme provided by the embodiment of the present disclosure can include the following beneficial effects.

It can be understood that the technical scheme provided by the present disclosure can construct a SWAT model of an area to be measured; construct a mathematical relation expression of vegetation potential transpiration, and introduce dynamic crop coefficients into the SWAT model; evaluate the results obtained by operating the SWAT model according to the measured evaporation data and leaf area index monitoring data obtained in advance, and calibrate crop growth parameters and evapotranspiration parameters; judge evaporation effectiveness according to a land cover type and a dynamic coverage calculation method, and carry out daily simulation to obtain a total amount of effective green water resources in the area to be measured. According to the technical scheme provided by the present disclosure, the dynamic crop coefficients are introduced, so that the model takes the characteristics of specific plant types into full account in the evapotranspiration simulation process, accurately reflects the actual evapotranspiration and leaf area index changes of different vegetation types in their respective development periods, and improves the accuracy of green water simulation and crop growth process simulation. On the basis of improving the accuracy of the crop growth process simulation, the dynamic coverage calculation method is introduced, the accuracy of evaluating effective green water resources is improved, the caliber of water resources evaluation is broadened, and more effective guidance is provided for more effective utilization and scientific management of water resources.

It is to be understood that both the foregoing general description and the following detailed description are exemplary and explanatory only, rather than limit the present disclosure.

### BRIEF DESCRIPTION OF THE DRAWINGS

The accompanying drawings, which are incorporated in and constitute a part of this specification, illustrate embodiments consistent with the present disclosure and together with the description, serve to explain the principles of the present disclosure.

FIG. 1 is a schematic diagram showing steps of a method of evaluating effective green water resources based on dynamic crop coefficients according to an exemplary embodiment.

FIG. 2 is a schematic diagram showing the comparison between simulated values and measured values of rice and sugarcane leaf area indices according to an exemplary embodiment.

FIG. 3 is a schematic diagram showing the comparison between measured and simulated monthly evapotranspiration processes of a calibration period and a verification period in Yulin and Bobai according to an exemplary embodiment.

FIG. 4 is a schematic diagram showing the comparison between measured and simulated monthly evapotranspiration processes of a calibration period and a verification period in Hepu according to an exemplary embodiment.

FIG. 5 is a spatial distribution diagram of effective and ineffective green water resources in a watershed according to an exemplary embodiment.

FIG. 6 is a schematic block diagram of a device of evaluating effective green water resources based on dynamic crop coefficients according to an exemplary embodiment.

### DETAILED DESCRIPTION OF THE EMBODIMENTS

Reference will now be made in detail to exemplary embodiments, examples of which are illustrated in the accompanying drawings. When the following description refers to the drawings, unless otherwise indicated, the same numbers in different drawings indicate the same or similar elements. The embodiments described in the following exemplary embodiments do not represent all embodiments consistent with the present disclosure. Rather, the embodiments are merely examples of devices and methods consistent with some aspects of the present disclosure as detailed in the appended claims.

### Embodiment 1

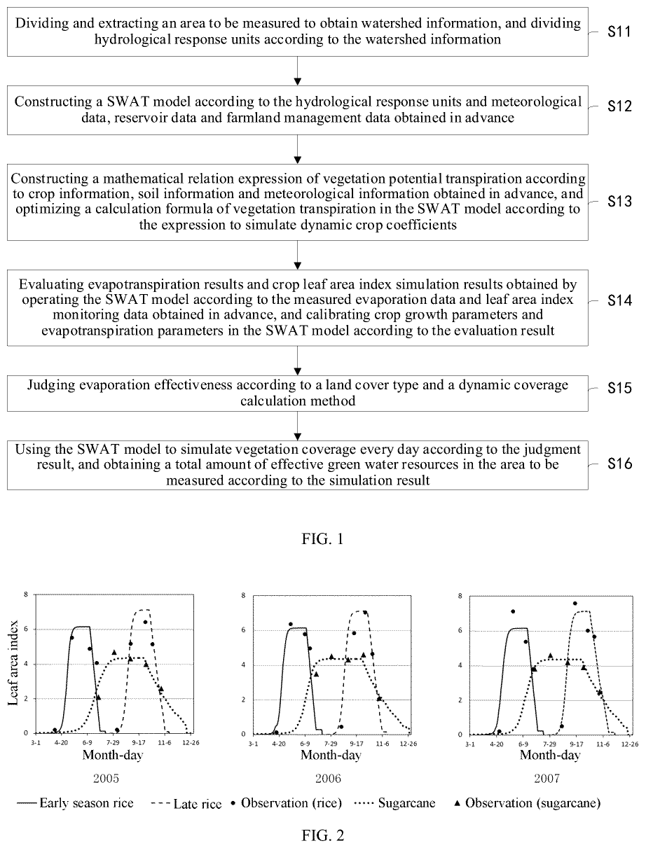

FIG. 1 is a schematic diagram showing steps of a method of evaluating effective green water resources based on dynamic crop coefficients according to an exemplary embodiment. Referring to FIG. 1, a method of evaluating effective green water resources based on dynamic crop coefficients is provided, comprising:
S11, dividing and extracting an area to be measured to obtain watershed information, and dividing hydrological response units according to the watershed information;
S12, constructing a SWAT model according to the hydrological response units and meteorological data, reservoir data and farmland management data obtained in advance;
S13, constructing a mathematical relation expression of vegetation potential transpiration according to crop information, soil information and meteorological information obtained in advance, and optimizing a calculation formula of vegetation transpiration in the SWAT model according to the expression to simulate dynamic crop coefficients;
S14, evaluating evapotranspiration results and crop leaf area index simulation results obtained by operating the SWAT model according to the measured evaporation data and leaf area index monitoring data obtained in advance, and calibrating crop growth parameters and evapotranspiration parameters in the SWAT model according to the evaluation result;
calibrating crop growth parameters and evapotranspiration parameters to realize the calibration of the results.
S15, judging evaporation effectiveness according to a land cover type and a dynamic coverage calculation method;
S16, using the SWAT model to simulate vegetation coverage every day according to the judgment result, and obtaining a total amount of effective green water resources in the area to be measured according to the simulation result.

It can be understood that the technical scheme provided by the present disclosure can construct a SWAT model of an area to be measured; construct a mathematical relation expression of vegetation potential transpiration, and introduce dynamic crop coefficients into the SWAT model; evaluate the results obtained by operating the SWAT model according to the measured evaporation data and leaf area index monitoring data obtained in advance, and calibrate crop growth parameters and evapotranspiration parameters; judge evaporation effectiveness according to a land cover type and a dynamic coverage calculation method, and carry out daily simulation to obtain a total amount of effective green water resources in the area to be measured. According to the technical scheme provided by the present disclosure, the dynamic crop coefficients are introduced, so that the model takes the characteristics of specific plant types into full account in the evapotranspiration simulation process, accurately reflects the actual evapotranspiration and leaf area index changes of different vegetation types in their respective development periods, and improves the accuracy of green water simulation and crop growth process simulation. On the basis of improving the accuracy of the crop growth process simulation, the dynamic coverage calculation method is introduced, the accuracy of evaluating effective green water resources is improved, the caliber of water resources evaluation is broadened, and more effective guidance is provided for more effective utilization and scientific management of water resources.

It should be noted that in step S11, dividing and extracting an area to be measured to obtain watershed information and dividing hydrological response units according to the watershed information comprises:
importing raster DEM data of the area to be measured into a geographic information system platform (ArcGIS) to obtain natural sub-watershed division data and river network water system data as watershed information;
importing the watershed information into an initial SWAT model, and dividing hydrological response units according to a land use type, a soil type and a slope type.

In step S12, the reservoir data comprises: reservoir capacity, outflow; etc. The farmland management data comprise: the crop vegetation type, vegetation time, harvest time, etc.

It should be noted that in step S13, constructing a mathematical relation expression of vegetation potential transpiration according to crop information, soil information and meteorological information obtained in advance comprises:

the mathematical relation expression of the vegetation potential transpiration is as follows:

$$EP = \{ \begin{matrix}{ET}_{0} \cdot {K}_{c ini} & phuc \leq {fr}_{phu 1} \\ {ET}_{0} \cdot {K}_{c mid} & {fr}_{phu 1} < phuc \leq {fr}_{phu 2} \\ {ET}_{0} \cdot {K}_{c end} & phuc > {fr}_{phu 2}\end{matrix}$$

where EP represents the vegetation potential transpiration in unit of mm; ET₀ represents reference evapotranspiration in unit of mm; K_{c ini}, K_{c mid} and K_{c end} represent crop coefficients of vegetation in an initial growth period, a rapid growth period and a late growth period, respectively; phuc represents a unit percentage of plant heat accumulation; fr_{gw1} and fr_{gw2} represent an accumulated temperature ratio corresponding to a first point and a second point on an optimal leaf area index curve, respectively.

Specifically, the calculation formula of K_{c ini} is as follows:

$${K}_{c ini} = \frac{TEW - ( TEW - REW ) \cdot exp [ \frac{1.15 \cdot {t}_{w} \cdot {ET}_{0} - REW}{TEW} \cdot ( 1 + \frac{REW}{TEW - REW} ) ]}{{t}_{w} \cdot {ET}_{0}}$$

where t_{w} represents an average interval between irrigation or precipitation; REW represents the amount of water evaporated in the control stage of atmospheric evaporation power, in unit of mm; TEW represents a total amount of water evaporated after precipitation or irrigation in unit of mm.

The expressions of REW and TEW are as follows:

$$REW = \{ \begin{matrix}20 - 0.15 \cdot Sand & if Sand \geq 80 \% \\ 11 - 0.06 \cdot Clay & if Clay > 50 \% \\ 8 + 0.08 \cdot Clay & if Sand < 80 \% and Clay < 50 \%\end{matrix}$$

$$TEW = \{ \begin{matrix}H \cdot ( {θ}_{FC} - 0.5 \cdot {θ}_{WP} ) & if {ET}_{0} \geq 5 mm / d \\ H \cdot ( {θ}_{FC} - 0.5 \cdot {θ}_{WP} ) \cdot \sqrt{\frac{{ET}_{0}}{5}} & if {ET}_{0} < 5 mm / d\end{matrix}$$

In the above formula, H represents the depth of a soil evaporation layer, which is usually in the range of 100 to 150 mm; θ_{Fc} and θ_{WP} are a field water holding capacity and a withering point water content of soil in the evaporation layer, respectively; Sand and Clay are the content of sand and the content of clay in soil, respectively.

The calculation formulas of K_{c mid} and K_{c end} are as follows:

$${K}_{c mid} = {K}_{cm} + [ 0.04 \cdot ( {v}_{2} - 2 ) - 0 \text{.004} \cdot ( {RH}_{min} - 45 ) ] \cdot {( \frac{h}{3} )}^{0.3}$$

$${K}_{c end} = \{ \begin{matrix}{K}_{cd} + [ 0.04 \cdot ( {v}_{2} - 2 ) - 0 \text{.004} \cdot ( {RH}_{min} - 45 ) ] \cdot {( \frac{h}{3} )}^{0.3} & if {K}_{cd} \geq 0.45 \\ {K}_{cd} & if {K}_{cd} < 0.45\end{matrix}$$

where v₂ represents an average wind speed at a height of 2 m in this growth stage; h represents an average height of vegetation in this growth stage; K_{cm} and K_{cd} represent reference values of crop coefficients provided by FAO in the middle and late stages of plant growth, respectively; RHₘᵢₙ represents an average value of the lowest relative humidity in this growth stage.

The specific expression of RHₘᵢₙ is as follows:

$${RH}_{min} = \frac{exp ( \frac{17.27 \cdot {T}_{min}}{{T}_{min} + 237.3} )}{exp ( \frac{17.27 \cdot {T}_{max}}{{T}_{max} + 237.3} )} \cdot 100$$

where Tₘₐₓ and Tₘᵢₙ represent a daily maximum temperature and a daily minimum temperature, respectively.

It should be noted that in step S14, evaluating evapotranspiration results and crop leaf area index simulation results obtained by operating the SWAT model according to the measured evaporation data and leaf area index monitoring data obtained in advance comprises:
based on the measured evaporation data and the leaf area index monitoring data obtained in advance, selecting a correlation coefficient and a Nash-Sutcliffe efficiency coefficient to evaluate the evapotranspiration results and the crop leaf area index simulation results obtained by operating the SWAT model.

In practice, the SWAT model which has been constructed is operated, and the simulation result of the evapotranspiration and the crop leaf area index is output. Based on the measured evaporation and leaf area index monitoring data, the correlation coefficient and the Nash-Sutcliffe efficiency coefficient are selected to evaluate the simulation effect, and the crop growth parameters and the evapotranspiration parameters of the model are calibrated.

It should be noted that the calibrated crop growth parameters comprise: the accumulated temperature ratio (FRGRW1) corresponding to the first point on the optimal leaf area index curve, the accumulated temperature ratio (FRGRW2) corresponding to the second point on the optimal leaf area index curve, a leaf area ratio (LAIMX1) corresponding to the first point on the optimal leaf area index curve, a leaf area ratio (LAIMX2) corresponding to the second point on the optimal leaf area index curve, and an accumulated temperature ratio (DLAI) corresponding to a potential maximum leaf area index (BLAI) and the time when an leaf area index begins to decline;
the calibrated evapotranspiration parameters comprise: a soil evaporation compensation factor (ESCO), a plant absorption compensation factor (EPCO), a shallow groundwater re-evaporation coefficient (GW_REVAP), a threshold depth (REVAPMN) of shallow aquifer “re-evaporation” or infiltration into a deep aquifer, an effective water capacity (SOL_AWC) of a soil layer and a vegetation interception capacity (CANMX).

It should be noted that in step S15, judging evaporation effectiveness according to a land cover type and a dynamic coverage calculation method comprises:
judging canopy interception evaporation, residential building interception evaporation, swamp or water area evaporation, phreatic water evapotranspiration and water participating in vegetation transpiration as effective green water resources;
using a dynamic coverage calculation method to calculate vegetation coverage of sparse forest land and non-high density grassland;
calculating soil evaporation between vegetation according to the vegetation coverage of sparse woodland and non-high density grassland.

In practice, the evaluation of effective green water resources of different land cover types is as follows:
the average storage variable of soil water storage for many years can be considered as zero, and the amount of green water resources is zero;
the canopy interception evaporation and the residential building interception evaporation are beneficial to maintaining the suitable physiological environment of vegetation and the suitable living environment of human beings, and are judged as effective green water resources;
the evaporations of swamp and water areas are beneficial to maintaining the ecological function of wetlands, both of which are judged as effective green water resources;
the phreatic water evapotranspiration is transformed from groundwater resources and is judged as an effective green water resource;
the water participating in vegetation transpiration directly participates in the growth process of vegetation and is judged as an effective green water resource;
for the soil evaporation between crops, forests, grasses and other vegetation (soil evaporation between plants), only a part belongs to effective green water, and its effectiveness depends on the vegetation coverage. Generally speaking, if the vegetation coverage is very low; which is far from enough to cover the space between vegetation, soil evaporation is basically ineffective for vegetation growth at this time, which is an ineffective green water resource. Therefore, all of the evaporations of inaccessible lands such as rock bare soil and sparse grassland are defined as ineffective green water resources. For sparse forest land and non-high density grassland, it is necessary to consider vegetation coverage to calculate an effective soil evaporation capacity.

It should be noted that the formula of the dynamic coverage calculation method is as follows:

$${a}_{i} = {LAI}_{i} / {LAI}_{mx}$$

where aᵢ represents vegetation coverage on an i-th day of a vegetation growing season; LAIᵢ represents a leaf area index of the i-th day of a vegetation growing season; LAIₘₓ indicates a maximum leaf area index during a vegetation growth period.

Specifically, for annual or perennial plants in grassland, shrub land and cultivated land, the calculation formula of the leaf area index is as follows:

$${LAI}_{i} = \{ \begin{matrix}{LAI}_{i - 1} + ( {fr}_{Lm , i} - {fr}_{Lm , i - 1} ) \cdot {LAI}_{mx} \cdot \{ 1 - exp [ 5 \cdot ( {LAI}_{i - 1} - {LAI}_{mx} ) ] \} \\ {LAI}_{mx} \\ {LAI}_{mx} \cdot \frac{1 - {fr}_{phu , i}}{1 - {fr}_{phu , s}}\end{matrix}$$

where fr_{phu,i} represents an accumulated heat unit fraction on the i-th day of the plant growing season; fr_{Lm,i} represents a maximum leaf area index fraction on the i-th day of the plant growing season. The calculation formula is as follows:

$${fr}_{Lm , i} = \frac{{fr}_{phu , i}}{{fr}_{phu , i} + exp ( {r}_{1} - {r}_{2} \cdot {fr}_{phu , i} )}$$

$${r}_{1} = ln ( \frac{{fr}_{phu 1}}{{LAI}_{mx 1}} - {fr}_{phu 1} ) + {r}_{2} \cdot {fr}_{phu 1}$$

$${r}_{2} = \frac{ln ( \frac{{fr}_{phu 1}}{{LAI}_{mx 1}} - {fr}_{phu 1} ) - ln ( \frac{{fr}_{phu 2}}{{LAI}_{mx 2}} - {fr}_{phu 2} )}{{fr}_{phu 2} - {fr}_{phu 1}}$$

where r₁ and r₂ represent a first form factor and a second form factor, respectively; LAIₘₓ₁ and LAIₘₓ₂ represent the leaf area ratio corresponding to the first point and the second point on the optimal leaf area index curve, respectively.

For the trees in the forest land, the calculation formula of the leaf area index is as follows:

$${LAI}_{i} = \{ \begin{matrix}{LAI}_{i - 1} + \frac{{yr}_{c}}{{yr}_{f}} \cdot ( {fr}_{Lm , i} - {fr}_{Lm , i - 1} ) \cdot {LAI}_{mx} \cdot \{ 1 - exp [ 5 \cdot ( {LAI}_{i - 1} - \frac{{yr}_{c}}{{yr}_{f}} \cdot {LAI}_{mx} ) ] \} \\ \frac{{yr}_{c}}{{yr}_{f}} \cdot {LAI}_{mx} \\ \frac{{yr}_{c}}{{yr}_{f}} \cdot {LAI}_{mx} \cdot \frac{1 - {fr}_{phu , i}}{1 - {fr}_{phu , s}}\end{matrix}$$

y_{rc} and y_{rf} represent the current age of the tree and the age of the tree, respectively.

It should be noted that using the SWAT model to simulate vegetation coverage every day and obtaining a total amount of effective green water resources in the area to be measured according to the simulation result comprises:
obtaining a total amount of effective green water resources in the area to be measured by the following formula:

$${Wgreen}_{a} = {\sum }_{i = 1}^{N} [ {W}_{can , i} + {Wwet}_{i} + {Wwt}_{i} + {Wgw}_{i} + {\sum }_{j = 1}^{M} ( {Wep}_{ij} + {a}_{ij} \cdot {Wes}_{ij} ) ]$$

where Wgreenₐ represents a total amount of effective green water resources in the area; W_{can,i} represents an intercepted evaporation capacity on the i-th day; Wwetᵢ indicates a wetland evaporation capacity on the i-th day; Wwti indicates a water area evaporation capacity on the i-th day; Wgwᵢ represents a phreatic water evaporation capacity on the i-th day; Wepᵢⱼ represents a vegetation transpiration capacity of a j-th vegetation on the i-th day; Wesᵢⱼ represents a soil evaporation capacity of the j-th vegetation on the i-th day; W_{un,i} represents a soil evaporation capacity of unused land on the i-th day; aᵢⱼ indicates vegetation coverage of the j-th vegetation on the i-th day; M represents a vegetation type in the watershed; and N represents a total number of days in a year.

It should be noted that the method further comprises:
obtaining a total amount of ineffective green water resources in the area to be measured by the following formula:

$${Wgreen}_{u} = {\sum }_{i = 1}^{N} [ {W}_{un , i} + {\sum }_{j = 1}^{M} ( 1 - {a}_{ij} ) \cdot {Wes}_{ij} ]$$

where Wgreenᵤ represents a total amount of ineffective green water resources in the area; W_{un,i} represent a soil evaporation capacity of the unused land on the i-th day.

This embodiment will be explained with a specific model application example.

1. Overview of the Study Area; Take Nanliujiang River Watershed in Guangxi as an Example to Illustrate that Nanliujiang River Flows Through Yulin, Qinzhou and Beihai.

### 2. Input of Basic Data:

Meteorological observation data, DEM data, land use data, soil type data, hydrology, evapotranspiration, reservoirs and other data are required for model construction.

Meteorological observation data; daily precipitation, temperature, wind speed, solar radiation and relative humidity of Lingshan, Yulin, Qinzhou and Beihai stations from 1990 to 2013; daily precipitation data of Pubei, Bobai and Hepu stations from 1990 to 2013.

Remote sensing data; the DEM uses 90 m grid data provided by Institute of Remote Sensing and Digital Earth, Chinese Academy of Sciences; land use data and soil type data are provided by Resource and Environment Science and Data Center.

Measured hydrological data: monthly flow data of Hengjiang, Bobai, Hejiang and Changle stations from 1990 to 2013 are used to verify runoff.

Measured evaporation data: monthly evaporation data of evaporating pan in Hengjiang, Bobai and Changle stations from 2000 to 2011 are used to verify evaporation.

Crop growth data: the experimental data of leaf area index of rice and sugarcane in Hepu irrigation area from 2005 to 2007 are used to verify crop growth simulation.

Reservoir data: data about a dead storage capacity, an utilizable capacity and a total storage capacity of 18 large-sized and medium-sized reservoirs; monthly inflow and outflow runoff data of three large reservoirs such as Xiaojiang Reservoir from 1990 to 2013.

### 3. Parameter Calibration and Model Verification:

The correlation coefficient R² and Nash-Sutcliffe efficiency coefficient Eₙₛ are selected to evaluate the simulation adaptability. It is generally considered that the simulation accuracy of the model is satisfactory when R² is greater than 0.6 and Eₙₛ is greater than 0.6.

### Plant Growth Simulation:

The main vegetation types in Nanliujiang River watershed are woodland and crops. The evapotranspiration of woodland almost belongs to effective green water, and the proportion of the grassland and shrub area is not high, so that the parameters of woodland, grassland and shrub are not calibrated any longer. The crops with the largest proportion are double-cropping rice and sugarcane. The sown area of double-cropping rice accounts for about 90% of the total sown area of crops, followed by sugarcane with the sown area accounting for more than 5%. The leaf area index of the above crops is low at the initial growth stage, and there are a lot of ineffective evapotranspiration, so that parameter calibration should be considered. Vegetables and other crop types account for very little area, which are not considered any longer. The corrected crop parameters are mainly six parameters, such as FRGRW1, FRGRW2 and LAIMX1, which are corrected in daily steps to minimize the simulation and observation residuals of the leaf area index of crops. Refer to Table 1 for the calibration results of main parameters of rice and sugarcane. The data comes from the observation experiment of rice and sugarcane growth in Hepu irrigation area from 2005 to 2007.

TABLE 1

| | early season rice | | late rice | | sugarcane | |
|---|---|---|---|---|---|---|
| | initial | final | initial | final | initial | final |
| parameters | value | value | value | value | value | value |
| FRGRW1 | 0.3 | 0.31 | 0.3 | 0.34 | 0.15 | 0.16 |
| FRGRW2 | 0.7 | 0.51 | 0.7 | 0.58 | 0.5 | 0.42 |
| LAIMX1 | 0.01 | 0.28 | 0.01 | 0.34 | 0.01 | 0.1 |
| LAIMX2 | 0.95 | 0.99 | 0.95 | 0.58 | 0.95 | 0.9 |
| BLAI | 5 | 6.30 | 5 | 7.28 | 6 | 4.5 |
| DLAI | 0.8 | 0.79 | 0.8 | 0.76 | 0.75 | 0.73 |

Referring to FIG. 2, it can be seen from FIG. 2 that the simulated values are well fitted with the measured values. Refer to Table 2 for the calibration and verification results of the leaf area index of main plants. The correlation coefficient R² and Nash efficiency coefficient Eₙₛ of the simulated values and the measured values during the calibration period are almost more than 0.90. It can be seen that the simulation effect of the leaf area index of rice and sugarcane is good. The Nash efficiency coefficient Eₙₛ and correlation coefficient R² during the verification period are also more than 0.8, reaching the required values.

TABLE 2

| | calibration (2005-2006) | | Verification (2007) | |
|---|---|---|---|---|
| crop type | R² | Eₙₛ | R² | Eₙₛ |
| early season | 0.83 | 0.83 | 0.82 | 0.87 |
| rice | | | | |
| late rice | 0.91 | 0.95 | 0.72 | 0.83 |
| sugarcane | 0.93 | 0.90 | 0.93 | 0.93 |

### Evapotranspiration Simulation:

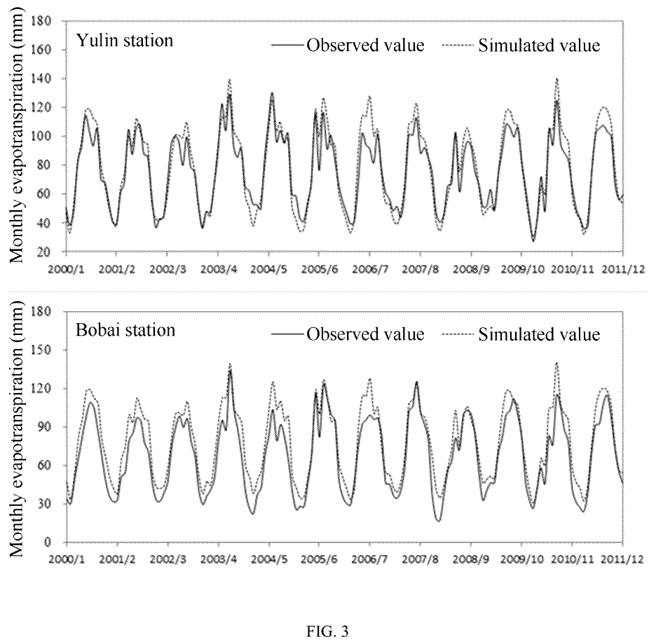

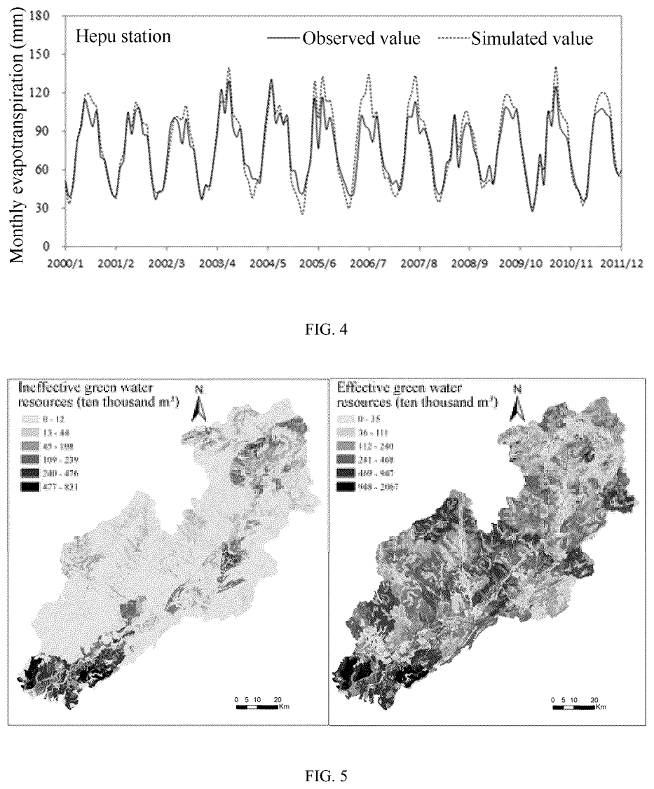

Six parameters, such as ESCO, EPCO and SOL_K, of Yulin, Bobai and Hepu stations are selected for calibration. Refer to Table 3 for the evaluation indexes of monthly evapotranspiration simulation results of the three stations. It can be seen from Table 3, FIG. 3 and FIG. 4 that the flow hydrographs of the simulated values and the measured values of monthly evapotranspiration are well fitted with each other. The correlation coefficient R² and the Nash efficiency coefficient Eₙₛ of the simulated values and the measured values of monthly runoff during the calibration period are basically more than 0.70. The Nash efficiency coefficient Eₙₛ and correlation coefficient R² of each hydrological station during the verification period are also basically more than 0.6, reaching the required values. Refer to Table 4 for the calibration results of sensitivity parameters of each station. In the table, r indicates that the existing parameter value is multiplied by (1+r), and v indicates that the parameter value is replaced by a given value.

TABLE 3

| | calibration (2000-2005) | | verification (2006-2011) | |
|---|---|---|---|---|
| station | R² | Eₙₛ | R² | Eₙₛ |
| Yulin | 0.73 | 0.70 | 0.61 | 0.59 |
| Bobai | 0.76 | 0.74 | 0.65 | 0.61 |
| Hepu | 0.87 | 0.85 | 0.82 | 0.79 |

TABLE 4

| | | calibration value | | |
|---|---|---|---|---|
| parameter | initial range | Hengjiang | Bobai | Changle |
| ESCO | v(0, 1) | 0.21 | 0.24 | 0.31 |
| EPCO | v(0, 1) | 0.73 | 0.65 | 0.86 |
| GW_REVAP | v(0.02, 2) | 0.48 | 0.30 | 1.67 |
| REVAPMN | v(0, 500) | 105.67 | 334.0 | 164.39 |
| SOL_AWC | r(−0.5, 0.5) | 0.28 | 0.27 | 0.28 |
| CANMX | r(−1, 1) | 0.89 | 0.69 | 0.12 |

### Evaluation of Effective Green Water Resources:

Refer to Table 5 for structural composition of green water in Nanliujiang River watershed. According to the summary simulation results in Table 5, the total amount of green water resources in Nanliujiang River watershed is 8.77 billion m³, in which vegetation transpiration accounts for the highest proportion (6.54 billion m³), accounting for 75.9% of the total amount of green water resources in the whole watershed. According to the division method of effective green water resources, based on the distribution of green water resources of different land use types and the coverage characteristics of different vegetation types, it is calculated that the effective green water resources in Nanliujiang River watershed are 7.75 billion m³, accounting for 90.1% of the total green water resources in the watershed, and the ineffective green water resources are 860 million m³, accounting for 9.9% of the total green water resources in the watershed. In terms of structural composition, the effective green water resources of forest land, fruit forest land and wetland are 4.63 billion m³, 180 million m³ and 160 million m³, respectively, accounting for 96.1%, 90.8% and 100% of their respective total green water resources. The effective green water resources of cultivated land are 2.24 billion m³, accounting for 79.7% of the total green water resource of cultivated land. The effective green water resources in grassland are 330 million m³, accounting for 86.1% of the total green water resources in shrub land.

TABLE 5

| | Total | | | | |
|---|---|---|---|---|---|
| Land | amount of | | Soil | Ineffective | Effective |
| cover | green | Vegetation | evapo- | green | green |
| type | water | transpiration | ration | water | water |
| residential | 2.6 | 1.7 | 0.8 | 0.5 | 2.1 |
| building | | | | | |
| Woodland | 48.2 | 41.1 | 7.0 | 1.9 | 46.3 |
| Fruit | 1.8 | 1.4 | 0.4 | 0.0 | 1.8 |
| forest | | | | | |
| land | | | | | |
| Rice | 19.1 | 12.7 | 6.4 | 3.3 | 15.8 |
| Sugarcane | 9.0 | 5.9 | 3.2 | 2.4 | 6.6 |
| Grassland | 3.8 | 2.6 | 1.2 | 0.5 | 3.3 |
| Wetland | 1.6 | 0.0 | 0.0 | 0.0 | 1.6 |
| In total | 86.1 | 65.4 | 19.1 | 8.6 | 77.5 |

As shown in FIG. 5, in terms of spatial distribution, the effective green water resources are mainly distributed in mountainous areas and estuary plain areas. The ineffective green water resources are mainly distributed in Yulin Basin in the upper reaches of Nanliujiang River watershed, Bobai Basin in the middle reaches and the estuary plain in the lower reaches, where the cultivated land is concentrated and the exposed area of topsoil is too large in the early stage of crop growth, resulting in more ineffective green water. In contrast, there is a little ineffective green water in mountainous areas, because the forest land is concentrated here, the vegetation coverage is large, and the green water utilization efficiency is high. It can be seen that increasing the forest area can effectively improve the amount of effective green water. The ineffective green water of the cultivated land in Nanliujiang River watershed is too large. It is suggested to take a series of measures such as intertillage, straw mulching and plastic film mulching to reduce direct sunlight, reduce soil surface water evaporation and improve the utilization efficiency of green water resources in farmland.

### Embodiment 2

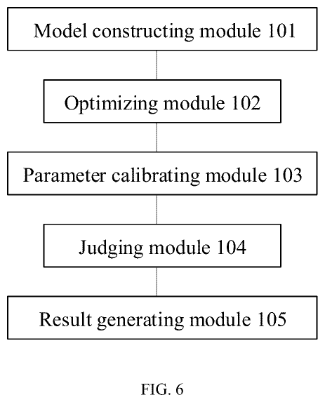

FIG. 6 is a schematic block diagram of a device of evaluating effective green water resources based on dynamic crop coefficients according to an exemplary embodiment. Referring to FIG. 6, a device of evaluating effective green water resources based on dynamic crop coefficients is provided, comprising:
a model constructing module 101, which is configured to divide and extract an area to be measured to obtain watershed information, divide hydrological response units according to the watershed information, and construct a SWAT model according to the hydrological response units and meteorological data, reservoir data and farmland management data obtained in advance;
an optimizing module 102, which is configured to construct a mathematical relation expression of vegetation potential transpiration according to crop information, soil information and meteorological information obtained in advance, and optimize a calculation formula of vegetation transpiration in the SWAT model according to the expression to simulate dynamic crop coefficients;
a parameter calibrating module 103, which is configured to evaluate evapotranspiration results and crop leaf area index simulation results obtained by operating the SWAT model according to the measured evaporation data and leaf area index monitoring data obtained in advance, and calibrate crop growth parameters and evapotranspiration parameters in the SWAT model according to the evaluation result;
a judging module 104, which is configured to judge evaporation effectiveness according to a land cover type and a dynamic coverage calculation method;
a result generating module 105, which is configured to use the SWAT model to simulate vegetation coverage every day according to the judgment result, and obtain a total amount of effective green water resources in the area to be measured according to the simulation result.

It can be understood that the technical scheme provided by the present disclosure can construct, by the model constructing module 101, a SWAT model of an area to be measured; construct, by the optimizing module 102, a mathematical relation expression of vegetation potential transpiration, and introduce dynamic crop coefficients into the SWAT model; evaluate, by the parameter calibrating module 103, the results obtained by operating the SWAT model according to the measured evaporation data and leaf area index monitoring data obtained in advance, and calibrate crop growth parameters and evapotranspiration parameters; judge, by the judging module 104, evaporation effectiveness according to a land cover type and a dynamic coverage calculation method; and carry out, by the result generating module 105, daily simulation to obtain a total amount of effective green water resources in the area to be measured. According to the technical scheme provided by the present disclosure, the dynamic crop coefficients are introduced, so that the model takes the characteristics of specific plant types into full account in the evapotranspiration simulation process, accurately reflects the actual evapotranspiration and leaf area index changes of different vegetation types in their respective development periods, and improves the accuracy of green water simulation and crop growth process simulation. On the basis of improving the accuracy of the crop growth process simulation, the dynamic coverage calculation method is introduced, the accuracy of evaluating effective green water resources is improved, the caliber of water resources evaluation is broadened, and more effective guidance is provided for more effective utilization and scientific management of water resources.

It can be understood that the same or similar parts in the above embodiments can refer to each other, and what is not explained in detail in some embodiments can refer to the same or similar parts in other embodiments.

It should be noted that in the description of the present disclosure, the terms “first” and “second” are only used for descriptive purposes and cannot be understood as indicating or implying relative importance. In addition, in the description of the present disclosure, unless otherwise specified, “a plurality of” means at least two.

Any process or method description in the flowchart or otherwise described herein can be understood as representing a module, segment or part of a code that includes one or more executable instructions for implementing the steps of the specific logical functions or process, and the scope of preferred embodiments of the present disclosure includes other implementations, in which functions can be performed out of the order shown or discussed, including in a substantially simultaneous manner or in the reverse order according to the functions involved, which should be understood by those skilled in the technical field to which embodiments of the present disclosure belong.

It should be understood that various parts of the present disclosure can be implemented in hardware, software, firmware or a combination thereof. In the above embodiments, a plurality of steps or methods can be implemented by software or firmware stored in a memory and executed by an appropriate instruction execution system. For example, if the steps or methods are implemented in hardware, as in another embodiment, the steps or methods can be implemented by any one of the following technologies or the combination thereof: a discrete logic circuit with a logic gate circuit for implementing a logic function on a data signal, an application specific integrated circuit with a suitable combinational logic gate circuit, a programmable gate array (PGA), a field programmable gate array (FPGA), and the like.

Those skilled in the art can understand that all or part of the steps carried by the above embodiment method can be completed by instructing related hardware through a program. The program can be stored in a computer-readable storage medium, and the program, when executed, comprises one of the steps of the method embodiment or the combination thereof.

In addition, each functional unit in each embodiment of the present disclosure may be integrated in one processing module, or each unit may exist physically alone, or two or more units may be integrated in one module. The above integrated modules can be implemented in the form of hardware or software functional modules. The integrated module can also be stored in a computer-readable storage medium if it is implemented in the form of a software functional module and sold or used as an independent product.

The storage medium mentioned above can be a read-only memory, a magnetic disk or an optical disk, etc.

In the description of this specification, descriptions referring to the terms “one embodiment”, “some embodiments”, “examples”, “specific examples” or “some examples” mean that the specific features, structures, materials or characteristics described in connection with this embodiment or example are included in at least one embodiment or example of the present disclosure. In this specification, the schematic expressions of the above terms do not necessarily refer to the same embodiment or example. Moreover, the specific features, structures, materials or characteristics described may be combined in any one or more embodiments or examples in a suitable manner.

Although the embodiments of the present disclosure have been shown and described above, it can be understood that the above embodiments are exemplary and cannot be understood as limitations of the present disclosure, and those skilled in the art can make changes, modifications, substitutions and variations to the above embodiments within the scope of the present disclosure.

What is claimed is:

1. A method of evaluating effective green water resources based on dynamic crop coefficients, comprising:
dividing and extracting an area to be measured to obtain watershed information, and dividing hydrological response units according to the watershed information;
constructing a SWAT model according to the hydrological response units and meteorological data, reservoir data and farmland management data obtained in advance;
constructing a mathematical relation expression of vegetation potential transpiration according to crop information, soil information and meteorological information obtained in advance, and optimizing a calculation formula of vegetation transpiration in the SWAT model according to the expression to simulate dynamic crop coefficients;
evaluating evapotranspiration results and crop leaf area index simulation results obtained by operating the SWAT model according to the measured evaporation data and leaf area index monitoring data obtained in advance, and calibrating crop growth parameters and evapotranspiration parameters in the SWAT model according to the evaluation result;
judging evaporation effectiveness according to a land cover type and a dynamic coverage calculation method;
using the SWAT model to simulate vegetation coverage every day according to the judgment result, and obtaining a total amount of effective green water resources in the area to be measured according to the simulation result.

2. The method according to claim 1, wherein dividing and extracting an area to be measured to obtain watershed information and dividing hydrological response units according to the watershed information comprises:
importing raster DEM data of the area to be measured into a geographic information system platform to obtain natural sub-watershed division data and river network water system data as watershed information;
importing the watershed information into an initial SWAT model, and dividing hydrological response units according to a land use type, a soil type and a slope type.

3. The method according to claim 1, wherein constructing a mathematical relation expression of vegetation potential transpiration according to crop information, soil information and meteorological information obtained in advance comprises:
the mathematical relation expression of the vegetation potential transpiration is as follows:
$$EP = \{ \begin{matrix}{ET}_{0} \cdot {K}_{c ini} & phuc \leq {fr}_{phu 1} \\ {ET}_{0} \cdot {K}_{c mid} & {fr}_{phu 1} < phuc \leq {fr}_{phu 2} \\ {ET}_{0} \cdot {K}_{c end} & phuc > {fr}_{phu 2}\end{matrix}$$
where EP represents the vegetation potential transpiration; ET₀ represents reference evapotranspiration; K_{c ini}, K_{c mid} and K_{c end} represent crop coefficients of vegetation in an initial growth period, a rapid growth period and a late growth period, respectively; phuc represents a unit percentage of plant heat accumulation; fr_{gw1} and fr_{gw2} represent an accumulated temperature ratio corresponding to a first point and a second point on an optimal leaf area index curve, respectively.

4. The method according to claim 1, wherein evaluating evapotranspiration results and crop leaf area index simulation results obtained by operating the SWAT model according to the measured evaporation data and leaf area index monitoring data obtained in advance comprises:
based on the measured evaporation data and the leaf area index monitoring data obtained in advance, selecting a correlation coefficient and a Nash-Sutcliffe efficiency coefficient to evaluate the evapotranspiration results and the crop leaf area index simulation results obtained by operating the SWAT model.

5. The method according to claim 4, wherein
the calibrated crop growth parameters comprise: the accumulated temperature ratio corresponding to the first point on the optimal leaf area index curve, the accumulated temperature ratio corresponding to the second point on the optimal leaf area index curve, a leaf area ratio corresponding to the first point on the optimal leaf area index curve, a leaf area ratio corresponding to the second point on the optimal leaf area index curve, and an accumulated temperature ratio corresponding to a potential maximum leaf area index and the time when an leaf area index begins to decline;
the calibrated evapotranspiration parameters comprise: a soil evaporation compensation factor, a plant absorption compensation factor, a shallow groundwater re-evaporation coefficient, a threshold depth of shallow aquifer “re-evaporation” or infiltration into a deep aquifer, an effective water capacity of a soil layer and a vegetation interception capacity.

6. The method according to claim 1, wherein judging evaporation effectiveness according to a land cover type and a dynamic coverage calculation method comprises:
judging canopy interception evaporation, residential building interception evaporation, swamp or water area evaporation, phreatic water evapotranspiration and water participating in vegetation transpiration as effective green water resources;
using a dynamic coverage calculation method to calculate vegetation coverage of sparse forest land and non-high density grassland;
calculating soil evaporation between vegetation according to the vegetation coverage of sparse woodland and non-high density grassland.

7. The method according to claim 6, wherein
the formula of the dynamic coverage calculation method is as follows:
$${a}_{i} = {LAI}_{i} / {LAI}_{mx}$$
where aᵢ represents vegetation coverage on an i-th day of a vegetation growing season; LAIᵢ represents a leaf area index of the i-th day of a vegetation growing season; LAIₘₓ indicates a maximum leaf area index during a vegetation growth period.

8. The method according to claim 7, wherein using the SWAT model to simulate vegetation coverage every day and obtaining a total amount of effective green water resources in the area to be measured according to the simulation result comprises:
obtaining a total amount of effective green water resources in the area to be measured by the following formula:
$${Wgreen}_{a} = {\sum }_{i = 1}^{N} [ {W}_{can , i} + {Wwet}_{i} + {Wwt}_{i} + {Wgw}_{i} + {\sum }_{j = 1}^{M} ( {Wep}_{ij} + {a}_{ij} \cdot {Wes}_{ij} ) ]$$
where Wgreenₐ represents a total amount of effective green water resources in the area; W_{can,i} represents an intercepted evaporation capacity on the i-th day; Wwetᵢ indicates a wetland evaporation capacity on the i-th day; Wwti indicates a water area evaporation capacity on the i-th day; Wgwᵢ represents a phreatic water evaporation capacity on the i-th day; Wepᵢⱼ represents a vegetation transpiration capacity of a j-th vegetation on the i-th day; Wesᵢⱼ represents a soil evaporation capacity of the j-th vegetation on the i-th day; W_{un,i} represents a soil evaporation capacity of unused land on the i-th day; aᵢⱼ indicates vegetation coverage of the j-th vegetation on the i-th day; M represents a vegetation type in the watershed; and N represents a total number of days in a year.

9. The method according to claim 8, further comprising:
obtaining a total amount of ineffective green water resources in the area to be measured by the following formula:
$${Wgreen}_{u} = {\sum }_{i = 1}^{N} [ {W}_{un , i} + {\sum }_{j = 1}^{M} ( 1 - {a}_{ij} ) \cdot {Wes}_{ij} ]$$
where Wgreenᵤ represents a total amount of ineffective green water resources in the area; W_{un,i} represent a soil evaporation capacity of the unused land on the i-th day.

10. A device of evaluating effective green water resources based on dynamic crop coefficients, comprising:
a model constructing module, which is configured to divide and extract an area to be measured to obtain watershed information, divide hydrological response units according to the watershed information, and construct a SWAT model according to the hydrological response units and meteorological data, reservoir data and farmland management data obtained in advance;
an optimizing module, which is configured to construct a mathematical relation expression of vegetation potential transpiration according to crop information, soil information and meteorological information obtained in advance, and optimize a calculation formula of vegetation transpiration in the SWAT model according to the expression to simulate dynamic crop coefficients;
a parameter calibrating module, which is configured to evaluate evapotranspiration results and crop leaf area index simulation results obtained by operating the SWAT model according to the measured evaporation data and leaf area index monitoring data obtained in advance, and calibrate crop growth parameters and evapotranspiration parameters in the SWAT model according to the evaluation result;
a judging module, which is configured to judge evaporation effectiveness according to a land cover type and a dynamic coverage calculation method;
a result generating module, which is configured to use the SWAT model to simulate vegetation coverage every day according to the judgment result, and obtain a total amount of effective green water resources in the area to be measured according to the simulation result.
